(19) United States
(12) Patent Application Publication
(10) Pub. No.: US 2008/0009560 A1
(43) Pub. Date: Jan. 10, 2008

(54) ARTICLES FOR REDUCING ATMOSPHERIC ODORS

(76) Inventor: Nicholas Douglas MCKAY JR., Atlanta, GA (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 11/767,088

(22) Filed: Jun. 22, 2007

Related U.S. Application Data
(60) Provisional application No. 60/815,945, filed on Jun. 23, 2006.
(60) Provisional application No. 60/828,696, filed on Oct. 9, 2006.
(60) Provisional application No. 60/885,666, filed on Jan. 19, 2007.

(51) Int. Cl. A61K 8/72

(52) U.S. Cl. 523/102

(57) ABSTRACT

Described herein are articles that reduce atmospheric odors. The articles are useful in removing and masking unpleasant odors that are present in households and other settings.

### CROSS REFERENCE TO RELATED APPLICATIONS

This application claims priority upon U.S. provisional application Ser. Nos. 60/815,945, filed Jun. 23, 2006; 60/828,696, filed Oct. 9, 2006; and 60/885,666, filed Jan. 19, 2007. These applications are hereby incorporated by reference in their entireties for all of their teachings.

### BACKGROUND

Particles that contain malodor exist throughout homes. Fibrous materials such as clothing and carpeting absorb these odors and provide an unpleasant environment. Typical methods of neutralizing malodors utilize air control devices that mask odors, such as air fresheners, potpourri, etc. Certain air control devices and chemicals also seek to neutralize malodorous molecules by reacting with them at the molecular level. Described herein are articles that reduce malodorous compounds in the atmosphere in a convenient manner.

### BRIEF DESCRIPTION OF THE DRAWINGS

The accompanying figures, which are incorporated in and constitute a part of this specification, illustrate several aspects described below.

FIG. 1 represents a longitudinal sectional view of a vacuum bag according to a first example embodiment of the present invention.

FIG. 2 is a perspective view of a lint roller according to a second example embodiment of the present invention.

FIGS. 3-16 show various storage containers with the odor-reducing materials incorporated therein.

FIGS. 17 and 18 show a hanging bag and hanger, respectively, with the odor-reducing materials incorporated therein.

FIGS. 19 and 20 show closet storage devices that can be prepared from the odor-reducing materials described herein.

FIGS. 21 and 22 shows a cross-section of extruded polymer with odor-reducing agent dispersed in the polymer.

FIG. 23 shows a storage container with an odor-reducing insert incorporated therein.

### SUMMARY

In accordance with the purposes of the disclosed materials, compounds, compositions, articles, devices, and methods, as embodied and broadly described herein are articles that reduce atmospheric odors. The articles have numerous applications in removing unpleasant odors present in households. Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

The materials, compounds, compositions, articles, devices, and methods described herein may be understood more readily by reference to the following detailed description of specific aspects of the disclosed subject matter and the Examples included therein and to the Figures.

Before the present materials, compounds, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the layer” includes mixtures of two or more such layers, and the like.

“Optional” or “optionally” means that the subsequently described event or circumstance can or cannot occur, and that the description includes instances where the event or circumstance occurs and instances where it does not.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers or prepared by methods known to those skilled in the art.

Reference will now be made in detail to specific aspects of the disclosed materials, compounds, compositions, articles, and methods, examples of which are illustrated in the accompanying Examples and Figures.

Described herein are articles for reducing atmospheric odors. The phrase “reducing atmospheric odors” is defined herein as reducing the concentration of malodorous molecules or masking the odor of malodorous molecules. The odor-reducing materials described herein are generally composed of a thermoplastic polymer, an odor-reducing agent, and a fragrance, each of which is discussed below.

I. Components of Odor-Reducing Materials

a. Thermoplastic Polymers

The selection of the thermoplastic polymer will vary depending upon the article to be produced. Thermoplastic materials can be formed into a number of different forms (e.g., films, fibers, shaped articles). Techniques known in the art can be used to tailor the polymer material for thermoplastic processing and particular end use of the article.

A variety of thermoplastic materials can be used herein. In general, the thermoplastic polymer does not absorb malodorous compounds. Such materials include, but are not limited to, polyesters such as poly(ethylene-co-terephthalate), poly(ethylene-co-1,4-naphthalene dicarboxylate), poly(butylene-co-terephthalate); poly(acrylonitrile-co-butadiene-co-styrene) polymers, acrylic polymers such as the polymethylmethacrylate, poly-n-butyl acrylate, poly(ethylene-co-acrylic acid), poly(ethylene-co-methacrylate); cellophane, cellulosics including cellulose acetate, cellulose acetate propionate, cellulose acetate butyrate and cellulose triacetate; fluoropolymers including polytetrafluoroethylene (Teflon), poly(ethylene-co-tetrafluoroethylene) copolymers, (tetrafluoroethylene-co-propylene) copolymers, polyvinyl fluoride polymers, etc., polyamides such as nylon 6, nylon 6,6; polycarbonates; polyimide materials; polyalkylenes including polyethylene, low density polyethylene, linear low density polyethylene, high density polyethylene, polypropylene, and biaxially oriented polypropylene; polystyrene, biaxially oriented polystyrene; vinyl films including polyvinyl chloride, (vinyl chloride-co-vinyl acetate) copolymers, polyvinylidene chloride, polyvinyl alcohol, (vinyl chloride-co-vinylidene dichloride) copolymers, specialty films including polysulfone, polyphenylene sulfide, polyphenylene oxide, polyesters, polyurethanes, and polyether ketones.

b. Odor-Reducing Agent

The odor-reducing agent is generally any compound that decreases the relative amount of malodorous compounds in the atmosphere relative to the amount of malodorous compounds present in the atmosphere absent the odor-reducing agent. In one aspect, the odor-reducing agents can be inorganic compounds, hydrazines, organic polymers, and mixtures thereof. Methods for incorporating odor-reducing agents into fibers are disclosed in U.S. Pat. No. 6,077,794 and U.S. Published Application No. 20040219126, which are incorporated by reference with respect to their disclosures of different types of deodorant compositions and methods for incorporating the deodorant compositions into fibrous materials. Examples of inorganic compounds include porous substances formed from silicon dioxide, titanium dioxide, zinc oxide, aluminum oxide or the like, porous substances such as zeolite, silica gel, active carbon or the like, or organic acid salts such as acetates or citrates, inorganic acid salts such as sulfates, phosphates, nitrates, chlorides, hydroxides, or oxides of metal such as copper, zinc, zirconium, silver, lead, iron, aluminum, calcium, magnesium, manganese, nickel, cobalt or the like, and the like. In another aspect, the odor-reducing agent is activated charcoal.

In another aspect, the odor-reducing agent is cyclodextrin. Cyclodextrin is a cyclic oligosaccharide consisting of at least six glucopyranose units joined by α(1″4) linkages. Although cyclodextrin with up to twelve glucose residues are known, the three most common homologs (α-cyclodextrin, β-cyclodextrin and γ-cyclodextrin) having 6, 7, and 8 residues, respectively, can be used herein.

Depending upon the selection of the thermoplastic polymer, the cyclodextrin can be modified so the cyclodextrin is compatible with the polymer. Cyclodextrin possesses hydroxyl groups that are capable of being converted to other groups. U.S. Pat. No. 5,882,565 discloses techniques for modifying or derivatizing cyclodextrin, the teachings of which are incorporated herein by reference. Examples of derivatized cyclodextrin include acylated cyclodextrin, alkylated cyclodextrin, cyclodextrin esters such as tosylates, mesylate and other related sulfo derivatives, hydrocarbyl-amino cyclodextrin, alkyl phosphono and alkyl phosphato cyclodextrin, imidazoyl substituted cyclodextrin, pyridine substituted cyclodextrin, carbonate and carbonate substituted cyclodextrin, carboxylic acid and related substituted cyclodextrin and others.

The mechanism by which the odor-reducing agent can reduce or eliminate odor will vary depending upon the selection and amount of agent that is used. For example, the odor-reducing agent can absorb (i.e., trap) odor-producing molecules. Alternatively, the odor-reducing agent can interact with the odor-producing molecule to render the odor-producing molecule non-odorous. The type of interaction that can occur between the odor-reducing agent and the odor-producing molecule can be covalent or non-covalent (e.g., ionic, electrostatic, dipole-dipole, etc.). The amount of odor-reducing agent can vary depending upon the selection of the odor-reducing agent (or combination of two or more odor-reducing agents) and the polymer used to produce the odor-reducing material.

c. Fragrances

The fragrances useful herein are compounds that mask the odor produced by certain atmospheric compounds. Thus, by masking, the malodorous molecules are not removed from the atmosphere but their odor is not detected by smell due to the pleasant odor of the fragrance. Suitable fragrances useful herein can be found in U.S. Pat. Nos. 4,145,184; 4,209,417; 4,515,705; and 4,152,272, all of which are incorporated herein by reference.

The fragrances can also be classified according to their volatility. The highly volatile, low boiling, perfume ingredients typically have boiling points of about 250° C. or lower. The moderately volatile fragrances are those having boiling points of from about 250° C. to about 300° C. The less volatile, high boiling, fragrances have boiling points of about 300° C. or higher.

Examples of the highly volatile, low boiling, fragrances include, but are not limited to, anethole, benzaldehyde, benzyl acetate, benzyl alcohol, benzyl formate, iso-bornyl acetate, camphene, cis-citral (neral), citronellal, citronellol, citronellyl acetate, paracymene, decanal, dihydrolinalool, dihydromyrcenol, dimethyl phenyl carbinol, eucalyptol, geranial, geraniol, geranyl acetate, geranyl nitrile, cis-3-hexenyl acetate, hydroxycitronellal, d-limonene, linalool, linalool oxide, linalyl acetate, linalyl propionate, methyl anthranilate, alpha-methyl ionone, methyl nonyl acetaldehyde, methyl phenyl carbinyl acetate, laevo-menthyl acetate, menthone, iso-menthone, myrcene, myrcenyl acetate, myrcenol, nerol, neryl acetate, nonyl acetate, phenyl ethyl alcohol, alpha-pinene, beta-pinene, gamma-terpinene, alpha-terpineol, beta-terpineol, terpinyl acetate, and vertenex (para-tertiary-butyl cyclohexyl acetate).

Examples of moderately volatile fragrances include, but are not limited to, amyl cinnamic aldehyde, iso-amyl salicylate, beta-caryophyllene, cedrene, cinnamic alcohol, coumarin, dimethyl benzyl carbinyl acetate, ethyl vanillin, eugenol, iso-eugenol, floracetate, heliotropine, 3-cis-hexenyl salicylate, hexyl salicylate, lilial (para-tertiarybutyl-alpha-methyl hydrocinnamic aldehyde), gamma-methyl ionone, nerolidol, patchouli alcohol, phenyl hexanol, beta-selinene, trichloromethyl phenyl carbinyl acetate, triethyl citrate, vanillin, and veratraldehyde. Cedarwood terpenes are composed mainly of alpha-cedrene, beta-cedrene, and other C₁₅H₂₄ sesquiterpenes.

Examples of the less volatile, high boiling, fragrances include, but are not limited to, benzophenone, benzyl salicylate, ethylene brassylate, galaxolide (1,3,4,6,7,8-hexahydro-4,6,6,7,8,8-hexamethyl-cyclopenta-gama-2-benzopyran), hexylcinnamic aldehyde, lyral (4-(4-hydroxy-4-methyl pentyl)-3-cyclohexene-10-carboxaldehyde), methyl cedrylone, methyl dihydro jasmonate, methyl-beta-naphthyl ketone, musk indanone, musk ketone, musk tibetene, and phenylethyl phenyl acetate. In another aspect, the fragrance is an essence oil.

II. Preparation of Films, Fibrous Sheets, and Shaped Articles

A variety of different articles can be produced with the odor-reducing materials described herein. The odor-reducing articles can be prepared using techniques known in the art. In one aspect, prior to extrusion and production of the odor-reducing material, the thermoplastic polymer and odor-reducing agent are intimately mixed to homogeneously disperse the odor-reducing agent throughout the thermoplastic polymer. This can be performed in dry form (i.e., powder), or solvents can be used to facilitate mixing. Alternatively, pellets or chips of the thermoplastic polymer can be coated with a solution of the odor-reducing agent and dried to remove any residual solvent prior to extrusion. Additional components such as surfactants and antimicrobial agents can be incorporated into the odor-reducing materials as needed.

Depending upon the selection of the thermoplastic polymer and odor-reducing agent, the odor-reducing agent can be covalently or non-covalently bonded to the thermoplastic polymer. For example, cyclodextrin has hydroxyl groups that are capable of reacting with functional groups present on the thermoplastic polymer to produce new covalent bonds. Alternatively, the thermoplastic polymer and/or the odor-reducing agent can be modified so that covalent bonds are formed between the thermoplastic polymer and/or the odor-reducing agent. The techniques disclosed in U.S. Pat. No. 7,166,671 for chemically-modifying polymers to attach cyclodextrin, which are incorporated by reference, can be used herein.

The odor-reducing articles produced from the odor-reducing materials can exist in a variety of forms, shapes, and sizes. In one aspect, the odor-reducing article is a film. The film can be used alone or in combination with other materials, fabrics or structural units produced by lamination, coextrusion or coating. It is also contemplated the film can have an adhesive on one or both sides.

In another aspect, the odor-reducing article can be a fibrous sheet. The fibers can be produced using techniques known in the art. For example, the fibers can be produced by meltblown and spunblown techniques. The fibers can be used to produce a nonwoven web of overlapping or interconnected fibers in a nonwoven manner. In one aspect, layers of fibers can be produced. For example, a web produced from meltblown fibers can be attached to a web of spunblown fibers. Variations of this layered system are possible (e.g., SMS, SMMS, etc, where S is spunblown fibers and M is meltblown fibers layered on each other). By varying the types of fibers, it is possible to increase surface area of the article, which ultimately can increase odor-reduction. For example, whereas it is not generally possible to reduce a spunblown fiber below 25 microns in diameter, meltblown fibers generally have smaller diameters around 4 microns, which dramatically increases overall surface area and therefore odor-reducing performance.

In another aspect, the odor-reducing material is formed into a shaped article from a mold. In one aspect, the shaped article for removing atmospheric odors is produced by the process comprising (1) extruding a first thermoplastic polymer comprising a first odor-reducing agent to produce a shaped article, and (2) applying a fragrance to the shaped article. In this aspect, the odor-reducing agent is homogeneously dispersed throughout the shaped article.

In another aspect, the shaped article is produced by coextruding a first thermoplastic polymer and a second thermoplastic polymer, wherein the first polymer comprises the odor-reducing agent homogeneously dispersed throughout the first polymer, wherein the first and second polymer are the same or different. In this aspect, the odor-reducing agent is homogeneously dispersed throughout the first polymer. In this aspect, a layer of polymer with odor-reducing agent is laminated on a polymer substrate. The thickness of the first and second polymer layers can vary depending upon the article to be produced. For example, if the extruded article is a trashcan, a thicker layer of first polymer is desirable so that more odor-reducing agent is present and protected from abrasions due to repeated use. The coextruded articles also have the benefit that not as much odor-reducing agent is needed to produce the shaped article with odor-reducing capabilities, which has cost benefits during production. Thus, when it is not necessary for the odor-reducing agent to be dispersed throughout the shaped article, the coextrusion techniques can be used to produce a laminate composed of the odor-reducing agent. The first and second polymers can be any of the thermoplastic polymers described above. Techniques for coextruding different polymers to produce layered articles are known in the art.

After the production of the odor-reducing article (e.g., film, fibrous sheet, shaped article), the article is contacted with a fragrance. Techniques for applying the fragrance to the article are known in the art, which include dipping, spraying, coating, and the like. In one aspect when an adhesive is applied to a film or fiber, the adhesive can have the fragrance mixed with the adhesive prior to applying the adhesive to the film or fiber. The amount of fragrance absorbed by the article will depend upon the selection of the thermoplastic polymer. Although the fragrance is a surface treatment, some fragrance can penetrate below the surface of the article.

It is also contemplated that additional odor-reducing agent can be applied to the article in addition to the fragrance. The additional odor-reducing agent can be the same or different from the odor-reducing agent that was extruded with the thermoplastic polymer. For example, FEBREEZE®, which includes a fragrance and cyclodextrin, can be applied to the odor-reducing articles described herein by spraying the solution on the article as needed. It is also contemplated that one or more fragrances can be applied to any of the articles described herein, wherein the article does not contain an odor-reducing agent as described herein.

The amount of fragrance and optional second odor-reducing agent can vary depending upon the composition of the article and the duration of odor reduction. With the odor-reducing agent incorporated throughout the material, it is possible to incorporate more fragrance into the material, where the fragrance interacts (e.g., complexes) with the odor-reducing agent.

It is also possible to control the release of fragrance by the selection of odor-reducing agent and fragrance. Not wishing to be bound by theory, the fragrance can complex with the odor-reducing agent. When a malodorous molecule interacts with the odor-reducing article, the molecule can displace the fragrance. Moreover, the odor-reducing agent can complex with the malodorous molecule. Thus, the fragrance is passively released from the article based upon an equilibrium reaction between the malodorous molecule and the odor-reducing agent, which can vary upon the concentration of malodorous molecules and temperature.

The duration of odor-reduction can vary depending upon the selection and amount of odor-reducing agent and fragrance that are used. Odor reduction can last from hours to several days, weeks, or months. The odor-reducing articles described herein are intended to provide long-term odor reduction. This is due in part to the odor-reducing agent incorporated throughout the material and not merely applied to the surface by coating or dipping.

The odor-reducing articles described above are produced by extruding the thermoplastic polymer with the odor-reducing agent to produce the article. In other aspects, the article is produced without the odor-reducing agent, and the odor-reducing agent and fragrance are subsequently applied to the article. In this aspect, a solution of the odor-reducing agent and fragrance can be produced at certain concentrations prior to application to the article. The selection of thermoplastic polymer can be selected so that the article readily absorbs the solution of the odor-reducing agent and fragrance, which results in more odor-reducing agent and fragrance incorporated into the article.

III. Application of Odor-Reducing Articles

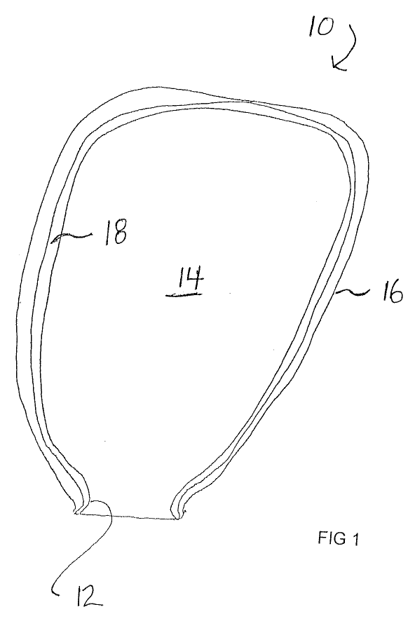

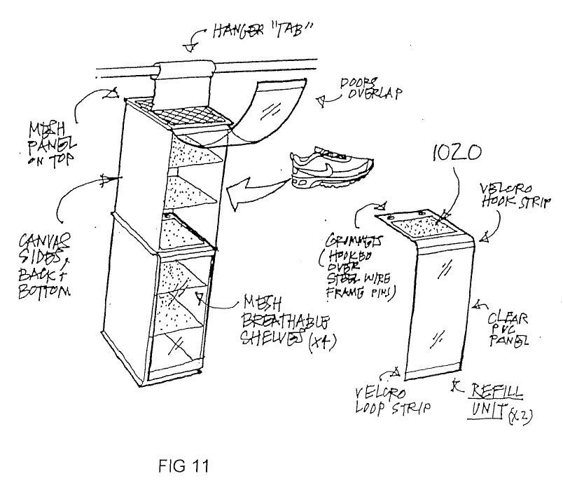

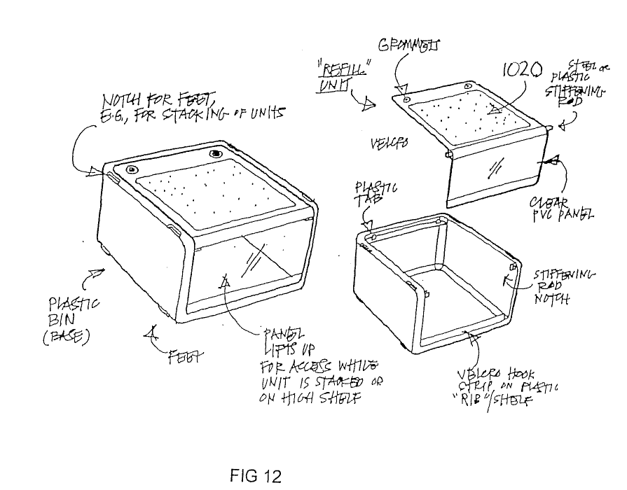

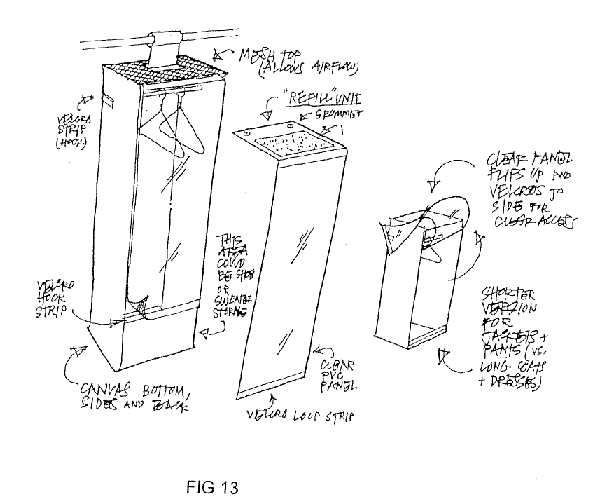

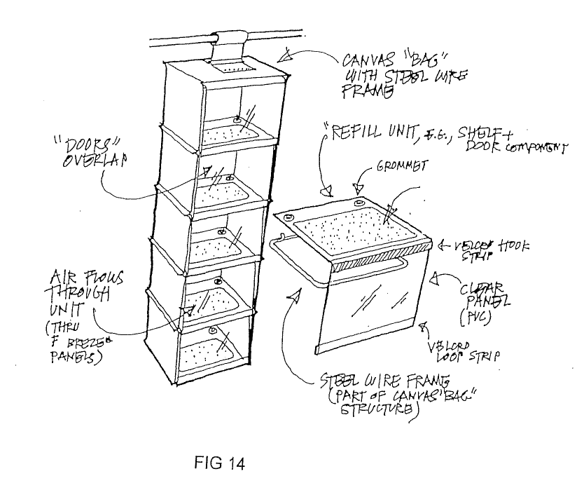

The odor-reducing articles described herein have numerous applications wherever undesirable odor persists. The articles are particularly useful in households where numerous sources of odor exist. FIGS. 1-22 provide numerous examples of how the articles can be used to reduce and mask odor.

a. Fibrous Articles

The fibrous materials described herein can be used to manufacture a number of articles. In one aspect, the fibrous materials can be used to produce articles including, but not limited to, vacuum bags and filters, laundry hampers, shelf or drawer liners, pet beds, lint rollers, clothing brushes, sheets, comforters, towels, electrostatic lint removers, lint traps, HVAC furnace filters, and dry cleaning bags

Vacuum cleaners are employed to remove dirt, dust and other debris. Such vacuum cleaners vary in construction, but many include a porous vacuum bag having an air inlet. The air inlet is connected to a vacuum cleaner attachment by a hose while a motor, upon activation, inducts air through the hose, into the bag and out through the bag where the air is finally exhausted. Some bags contain lining(s), which function as filters within the interior of the bag, such as a woven or non-woven liner, that prevent the passage of extremely small particles of dirt and debris through the liner, thus entrapping the debris within the interior of the bag. Some bags contain only the bag material itself, without such liners, with the bag material performing the filtration function, albeit not as effectively as lined bags.

One problem with these previously known vacuum cleaners is that the air exhausted from the vacuum cleaner bag exhibits an unpleasant odor. This unpleasant odor may arise from the material, which is actually entrapped within the interior of the vacuum cleaner bag.

With reference to FIG. 1, a first example embodiment of a vacuum cleaner bag 10 of the present invention is shown having an air inlet 12 and defining an interior chamber 14. The air inlet 12 is connected via a hose (not shown) to a vacuum attachment in the conventional fashion. The vacuum bag 10 comprises an outer covering 16 which may be constructed of any air-permeable material, such as paper. Additionally, a debris filter 18 may be used and is provided within the interior of the bag 10 inside of the covering 16 and around the interior chamber 14. The filter 18 may be constructed of any of the fibers described above. In one aspect, odor-reducing agent is cyclodextrin. The filter 18 is designed so that it is permeable to air, but not to debris, at least of a size greater than a predetermined amount. Consequently, as air is inducted into the interior 14 of the bag 10, air passes through both the filter material 18 and covering 16 whereas dirt, debris and other material remains entrapped within the chamber 14. The malodorous molecules are trapped within the interior chamber 14 of the bag 10 and do not exhaust through the filter material 18 and covering 16.

In another aspect, fibers described herein can be used to produce furnace and/or air conditioner filters to prevent malodors from circulating in the building during operation of either the heating or air conditioning system.

In one aspect, the fibrous materials can be used in the manufacture of lint removers. Lint removers are commonly used to remove material such as lint, dust, dandruff, pet hair, etc. from clothing and other upholstered items (e.g., couches) and flooring. Such materials can impart an unpleasant odor to the fabric. Additionally, the fabric may contain malodor such as smoke odor.

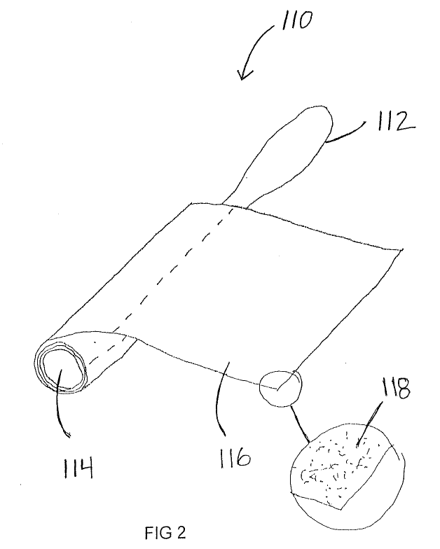

With reference to FIG. 2, a second example embodiment of a lint roller 110 is shown. The lint roller 110 has a handle 112 for grasping with the human hand, a roller 114 that rotates relative to the handle, and adhesive sheets 116 on the roller. The sheets are prepared by the fibers described above with the odor-reducing agent and fragrance. The sheets 116 are scored (perforated) so that after a sheet has been used and its adhesive properties have diminished, the sheet can be pulled to unroll it from the roller (as depicted in FIG. 2), and it can be torn away to expose the next sheet for subsequent use. The adhesive applied onto the sheets 116 can be a conventional adhesive used in commercially available lint rollers. It will be understood that the sheets can be configured in other ways so that a sheet is exposed for use, then can be removed after use to expose a subsequent sheet for subsequent use.

In use, as the exposed sheet is rolled over a surface, the adhesive collects lint and other matter from surface, in the same manner as a conventional lint roller. At the same time, the odor-reducing agent and/or fragrance can be dispensed onto the surface to reduce any odors and/or to apply a pleasant scent. In the alternative, the malodorous molecules can be absorbed by the odor-reducing agent present on the sheet when the sheet comes into contact with the intended surface.

In alternative embodiments, instead of a lint roller, another lint removing device is provided. For example, a lint brush with a felt material for removing lint can have the felt produced from fibers described herein. The lint brush is made of low-cost materials so that it is disposable after the effective lifespan of the odor-reducing agent (i.e., after all of the agent has been applied). In other alternative embodiments, lint shavers or electrostatic lint removers are provided with a dispenser system for delivering the odor-reducing agent to the fabric during use. The dispenser can be a strip of fabric or other material that is impregnated or saturated with odor-reducing agent, a storage container with nozzle and pump system that sprays the agent in response to the lint removal head engaging the fabric, or another conventional dispensing system. In still another alternative embodiment, a lint trap (e.g., on a washing machine) is impregnated or saturated with odor-reducing agent.

Accordingly, the lint roller described herein provides advantages over known devices. Generally described, in some of its various forms the invention includes a lint removal device having a supply of an odor-reducing agent that is applied during use to remove lint and other matter from a surface. Thus, the lint removal devices reduce odors and/or add scents in addition to removing lint and other matter.

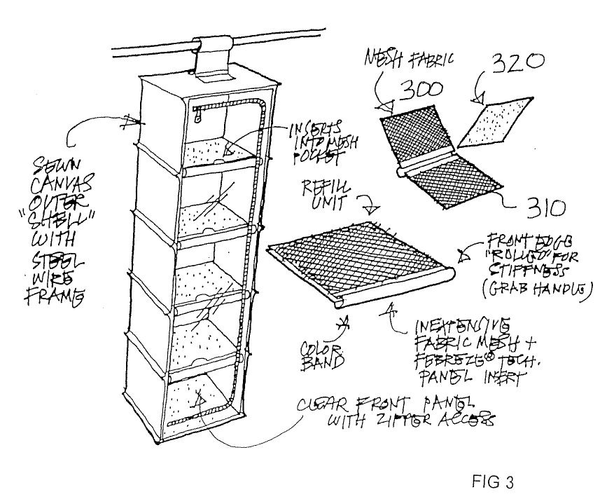

FIGS. 3-7 provide numerous embodiments with respect to using the fibrous sheets described herein as inserts for reducing atmospheric odors. The inserts can be constructed in a number of different shapes and sizes. In one aspect, the insert can have a frame to provide rigidity and permit easy insertion into a device such as a storage container. The frame can be any durable material such as, for example, cardboard, plastic, and paper. It is desirable that the frame be constructed of a material that does not absorb malodorous compounds. In some aspects, metal ribs in varying patterns can be attached to the frame of the insert to provide additional support and rigidity. In other aspects, the insert can be composed of a solid backing made of durable material (e.g., plastic, cardboard) with the fibrous sheet attached to the backing with an adhesive.

The insert can be attached or secured to an article by a variety of techniques. When the insert is a card, it can be inserted into a pocket of the article, where the pocket is a netting or mesh that permits airflow. Alternatively, the insert can be attached to the article by conventional fasteners such as adhesives, zippers, snaps, buttons, etc. The insert is attached so that it can easily be removed and replaced with another insert.

In the case when the insert is re-usable, the insert can be sprayed with additional fragrance and optional odor-reducing agent to regenerate the odor-reducing capabilities of the insert. When an insert is to be stored for future use, the insert can be placed in a container such as, for example, a vinyl zip-lock bag or wrapped in foil, which prevents airflow and preserves the insert.

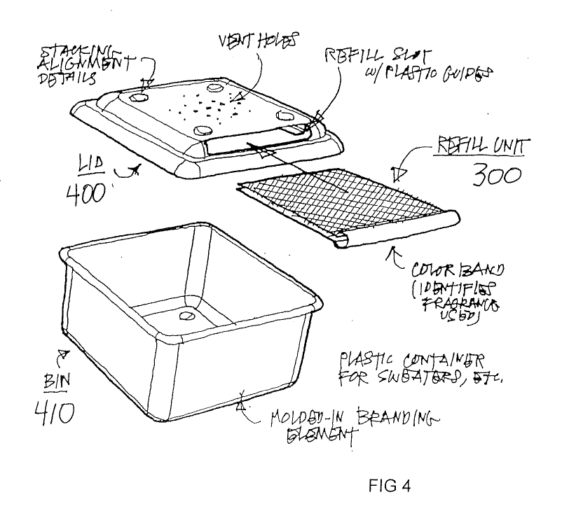

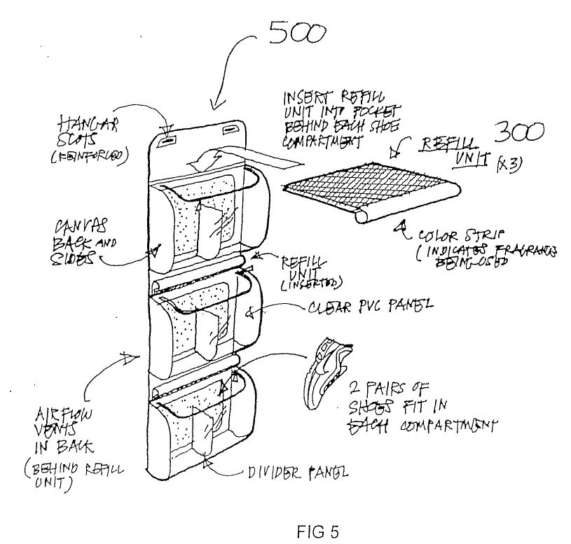

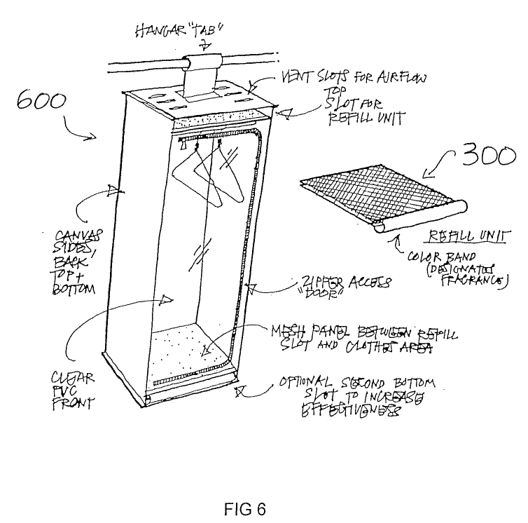

Referring to FIGS. 3-6, mesh insert 300 is composed of a two panels of mesh fabric 310 that can secure an odor-reducing fibrous sheet 320. Once the sheet is secured in the mesh insert, the insert can be placed in a number of different containers that store or hold articles that produce odors. For example, the insert 300 can be inserted into the lid 400 of a storage bin 410 (FIG. 4). In other aspects, insert 300 can be used in shoe rack 500 in FIG. 5 and hanging closet 600 in FIG. 6.

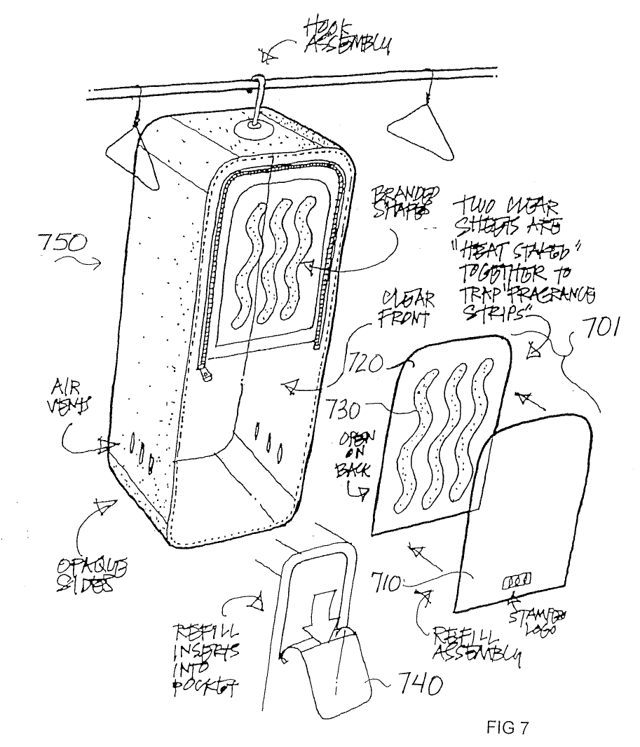

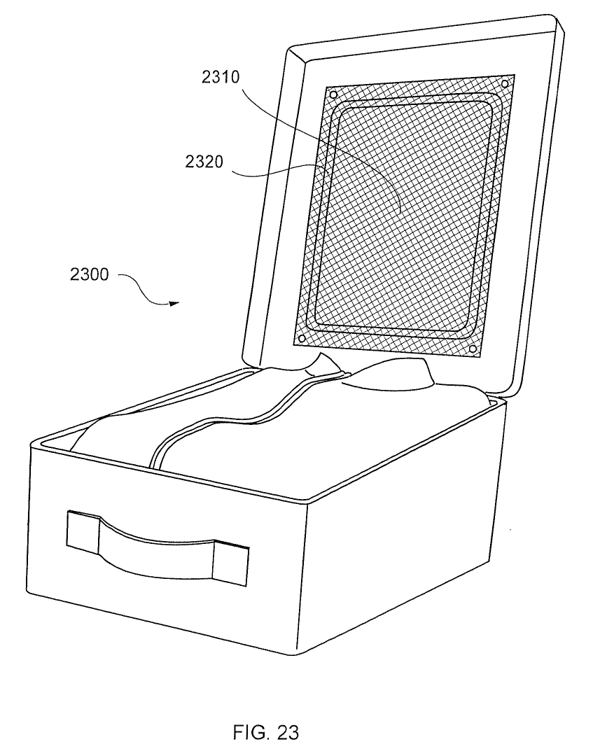

FIG. 7 depicts another aspect of the insert. The insert 700 is composed of clear sheet 710 (e.g., PEVA, PVA, PVC, etc.) sealed to insert 720, where the odor-reducing material is depicted as 730. The clear sheet 710 prevents any fragrance from escaping the insert and remaining in the container. In one aspect, the frame can be made of any inexpensive, durable material. Insert 700 can be placed in flap 740 of the storage container 750. FIG. 23 depicts another aspect of the insert. In this aspect, storage container 2300 has a net or mesh 2310 that can receive insert 2320, where the insert has a frame. Although not shown in FIG. 23, a flap on the other side of the insert can be opened to permit airflow into the storage device. Thus, in this aspect, the insert is between to pieces of netting or mesh.

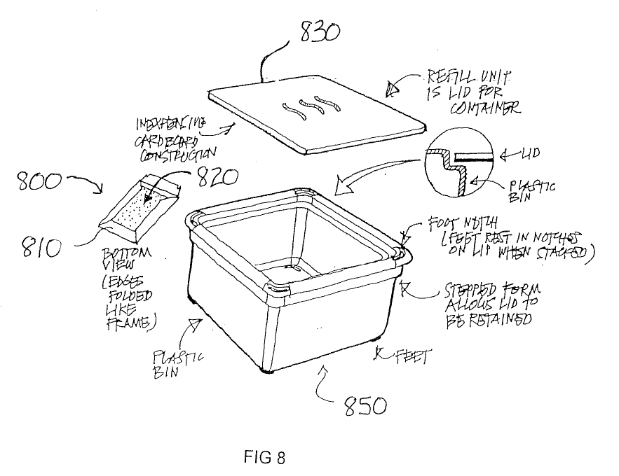

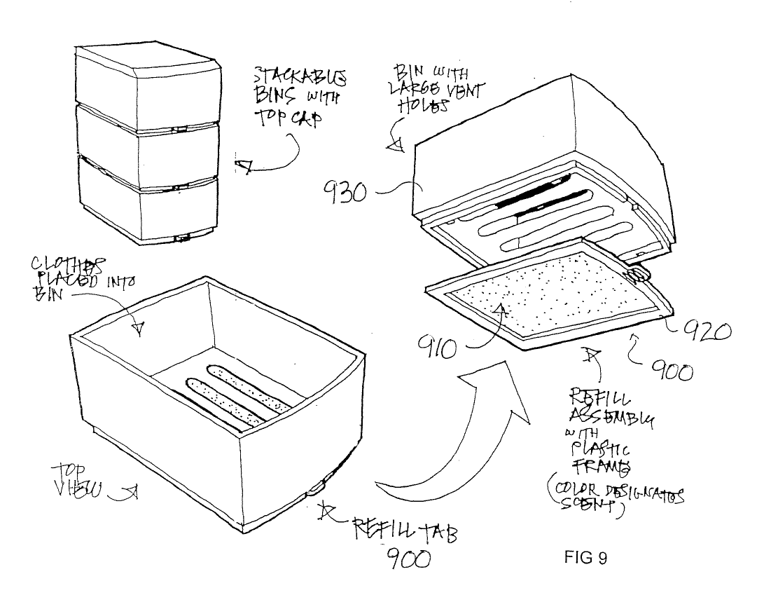

FIGS. 8 and 9 depict the use of fibrous materials incorporated into lid or container bottom, respectively, used to construct the storage container. In FIG. 8, insert 800 is composed of fibrous material 820 secured in a durable material 810. The insert can then be placed in lid 830. In FIG. 9, assembly 900 is composed of fibrous material 910 secured to frame 920. The assembly 900 can be attached to the bottom of storage bin 930.

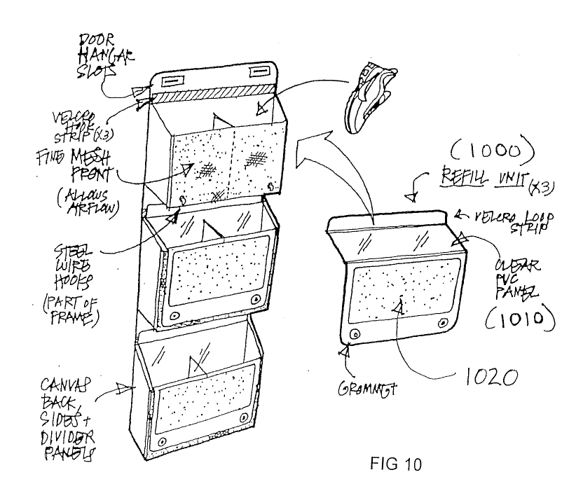

In other aspects, the sheets described herein can be incorporated into panels that can be subsequently attached to storage devices. This is depicted in FIGS. 10-14. Referring to FIG. 10, panel 1000 is constructed of a material such as clear PVC 1010. On the backside of the panel is an odor-reducing sheet 1020 described herein attached to the panel. The sheet can be attached via an adhesive, or it can be inserted into a pocket that can be readily replaced. The panels can be attached to container by Velcro, pins, hooks, grommets, or other attachment devices.

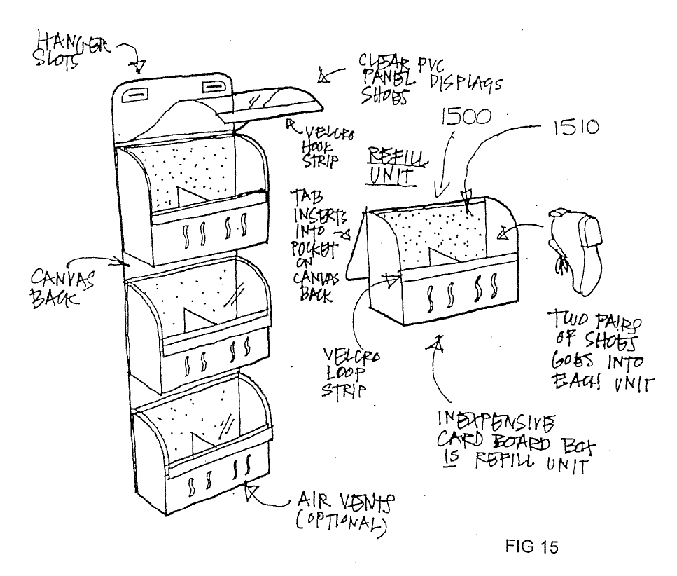

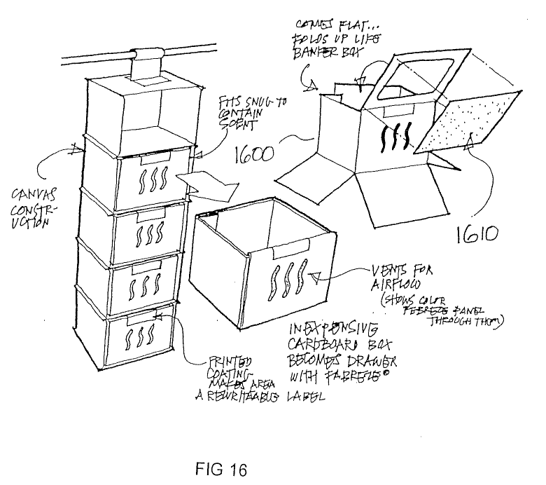

FIGS. 15 and 16 show embodiments where the fibrous sheet is adhered to the storage container. In FIG. 15, sheet 1510 is bonded to the container 1500. In FIG. 16, sheet 1610 is adhered to the backside of box 1600. The box can be constructed of cardboard or other materials that can be readily folded. Techniques and adhesives known in the art can be used to adhere the sheet to the storage device.

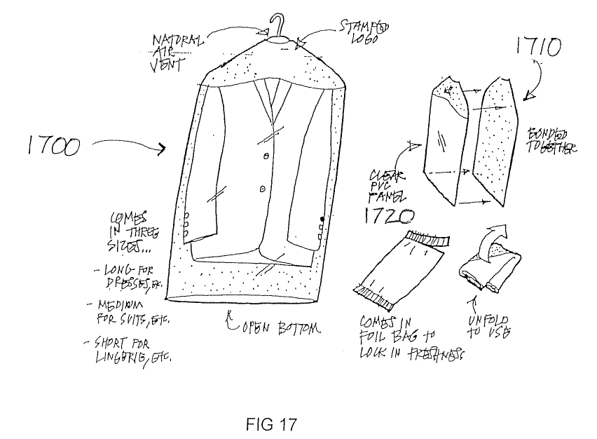

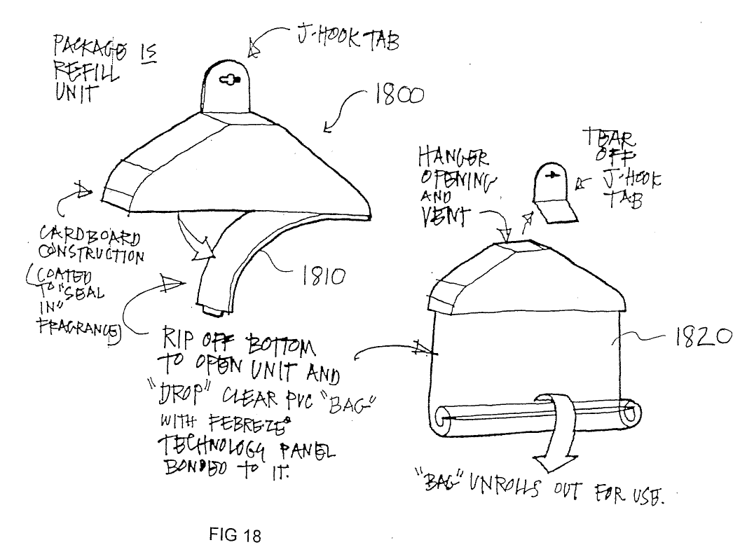

FIGS. 17 and 18 depict additional embodiments with respect to the sheets described herein. In FIG. 17, a clear sheet of PVC or other material 1720 is bonded or adhered to odor-reducing sheet 1710 to produce a hanging bag 1700 useful in reducing odors from clothes. FIG. 18 shows an extension of the hanging bag depicted in FIG. 17. Package 1800 has a seal 1810 that can be removed from the package. When the seal is removed, the hanging bag 1820 rolls out. The hanging bag 1820 is constructed in the same manner as the hanging bag 1700 depicted in FIG. 17. The hanging bag is useful in household and commercial applications (e.g., removing odors from clothing produced from chemicals used in dry cleaning).

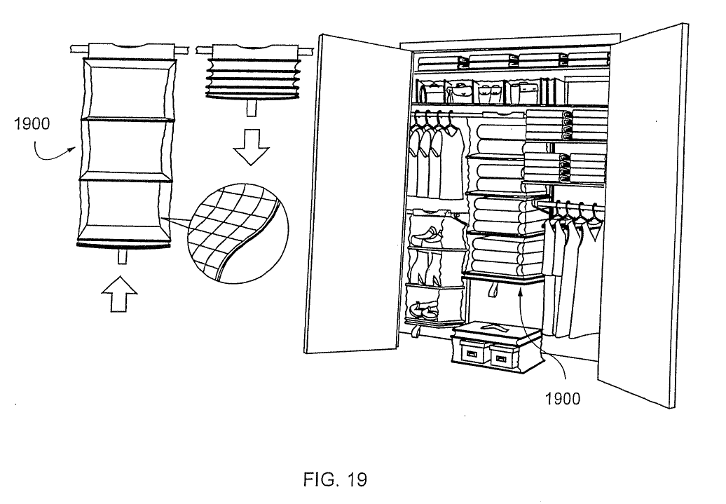

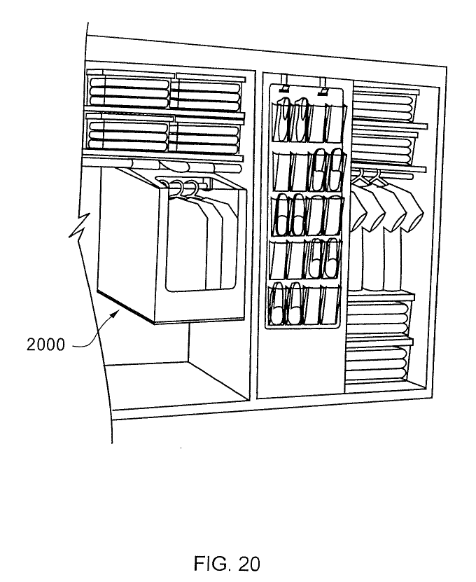

In certain aspects, the storage container is composed of a fabric such as canvas. In these aspects, the sheet can be attached to the fabric by Velcro, adhesive, or other techniques. Examples of these storage devices are shown in FIGS. 19 and 20, which depicts hanging storage containers 1900 and 2000, respectively. In certain aspects, it is desirable the fabric is not permeable to air, thus preserving the fragrant atmosphere within the container if the container is intended to be closed (e.g., 2000 in FIG. 20). Alternatively, the fabric can be composed of a non-woven material that permits some airflow (i.e., breathable).

b. Shaped Articles

In other aspects, the odor-reducing materials described herein can be extruded to produce shaped articles. Using techniques known in the art, a variety of different articles can be produced depending upon the end-use of the article. For example, injection or blow molding can be used to manufacture the shaped articles. The amount of thermoplastic polymer, odor-reducing agent, and fragrance used to produce the shaped articles can vary. For example, if the walls of the container are relatively thin, the wall can be composed solely of thermoplastic polymer, odor-reducing agent, and fragrance. Alternatively, if the wall of the container is thick, the odor-reducing materials described herein can be co-extruded so that a layer of odor-reducing material is laminated on the surface of another non-odor reducing polymer. In one aspect, a layered system can be produced with a polymer layer sandwiched between two odor-reducing polymer materials described herein. This aspect can be useful in producing indoor trashcans, where the entire article is essentially laminated with the odor-reducing composition. In general, it is desirable to select polymers that do not absorb malodorous molecules.

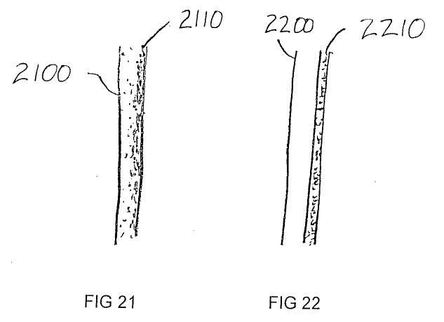

The shaped article (e.g., a storage device) has at least one wall composed of the odor-reducing composition. Referring to FIG. 21, in one aspect, the storage device wall 2100 can be made of polyethylene, polypropylene, hi-density polypropylene or another plastic, with the odor-reducing agent 2110 combined or impregnated into the plastic prior to molding, extruding, or otherwise fabricating the storage device.

Referring to FIG. 22, in another aspect, the wall of the storage device 2200 is made of polyethylene, polypropylene, hi-density polypropylene or another plastic, with the odor-reducing agent 2210 coated, sprayed, or otherwise applied onto the plastic after molding, extruding, or otherwise fabricating the storage device. Although this approach involves an additional manufacturing step, a significantly smaller amount of the odor-reducing agent can be used (e.g., by only applying the agent to the surface of the wall and dispersed throughout the entire wall) while still providing good odor-reducing properties.

The shaped articles can be produced in a variety of different shapes and sizes depending upon the items to be stored. A commercially available box, such as that sold under the brand name “TUPPERWARE” by Tupperware Corporation (Orlando, Fla.), can be adapted for use in the present invention. The shaped article can be provided with a size and shape selected for storing a variety personal or household items such as one or more articles of clothing (e.g., suits, sweaters), food products (e.g., fish or other malodorous foodstuffs), trash and debris (e.g., a trash bag or can) or any other personal or household item that is commonly stored and is prone to release an odor or to take on an odor. When the article is a storage container, the container can be configured with a lid so that an air-tight seal is formed, which prevents odors from escaping and maintaining a fragrant atmosphere within the container.

It is contemplated the fibrous sheets and shaped articles described herein can combined in a number of different ways to remove and mask odors. For example, referring to FIG. 8, one of the interior walls of the storage bin 850 can be composed of odor-reducing agent extruded (or co-extruded to produce a laminate) while the lid 830 is configured to hold an insert 800 as described above. Using this approach, it is possible to achieve consistent, long-term reduction of odors.

The storage devices produced by the odor-reducing materials include, but are not limited to, a closet storage device such as a shelf, rack, shoe tree, hanger, or hook, a general purpose utility storage device such as a tray or box, or another conventional container or other storage device typically used for storing items in the attic or garage. In yet other alternative embodiments contemplated by and included in the present invention, the storage device is provided in the form of soft-sided clothing storage devices like those that hang over a closet door or from a closet rod and that are constructed of woven or non-woven materials like cotton or breathable mesh. And in still other alternative embodiments contemplated by and included in the present invention, the storage device includes more than one odor-reducing agent, with the agents selected for targeted odors, for example, zinc for human body odor and cyclodextrin for other odors. The container may be made of a material other than plastic, such as wood, fiberglass, ceramic, metal, wire, etc., which has a sheet or extruded article incorporated in the container (e.g., a pocket to receive an insert).

While the invention has been shown and described in exemplary forms, it will be apparent to those skilled in the art that many modifications, additions, and deletions can be made therein without departing from the spirit and scope of the invention as defined by the following claims.

What is claimed is:

1. A film or fibrous sheet for reducing atmospheric odors produced by the process comprising applying a fragrance to the film or sheet, wherein the film and sheet is produced by extruding a thermoplastic polymer and a first odor-reducing agent.

2. The film or sheet of claim 1, wherein the odor-reducing agent is homogeneously dispersed throughout the thermoplastic polymer.

3. The film or sheet of claim 1, wherein the fibrous sheet comprises woven fibers, non-woven fiber, or a combination thereof.

4. The film or sheet of claim 1, wherein the polymer comprises a poly(acrylonitrile-co-butadiene-co-styrene) polymer, an acrylic polymer, cellophane, a cellulosic, a fluoropolymer, a polyamide, a polycarbonate, a polyester, a polyimide, a polyethylene, a polypropylene, a polystyrene, a vinyl film, or any combination thereof.

5. The film or sheet of claim 1, wherein polymer comprises a polyalkylene homopolymer or copolymer.

6. The film or sheet of claim 1, wherein the polymer comprises polypropylene.

7. The film or sheet of claim 1, wherein the first odor-reducing agent comprises an inorganic compound, a hydrazine, or an organic polymer.

8. The film or sheet of claim 1, wherein the first odor-reducing agent comprises α-cyclodextrin, β-cyclodextrin, γ-cyclodextrin, or any combination thereof.

9. The film or sheet of claim 1, wherein the fragrance comprises anethole, benzaldehyde, benzyl acetate, benzyl alcohol, benzyl formate, iso-bornyl acetate, camphene, cis-citral (neral), citronellal, citronellol, citronellyl acetate, paracymene, decanal, dihydrolinalool, dihydromyrcenol, dimethyl phenyl carbinol, eucalyptol, geranial, geraniol, geranyl acetate, geranyl nitrile, cis-3-hexenyl acetate, hydroxycitronellal, d-limonene, linalool, linalool oxide, linalyl acetate, linalyl propionate, methyl anthranilate, alpha-methyl ionone, methyl nonyl acetaldehyde, methyl phenyl carbinyl acetate, laevo-menthyl acetate, menthone, iso-menthone, myrcene, myrcenyl acetate, myrcenol, nerol, neryl acetate, nonyl acetate, phenyl ethyl alcohol, alpha-pinene, beta-pinene, gamma-terpinene, alpha-terpineol, beta-terpineol, terpinyl acetate, vertenex (para-tertiary-butyl cyclohexyl acetate), amyl cinnamic aldehyde, iso-amyl salicylate, beta-caryophyllene, cedrene, cinnamic alcohol, coumarin, dimethyl benzyl carbinyl acetate, ethyl vanillin, eugenol, iso-eugenol, flor acetate, heliotropine, 3-cis-hexenyl salicylate, hexyl salicylate, lilial (para-tertiarybutyl-alpha-methyl hydrocinnamic aldehyde), gamma-methyl ionone, nerolidol, patchouli alcohol, phenyl hexanol, beta-selinene, trichloromethyl phenyl carbinyl acetate, triethyl citrate, vanillin, veratraldehyde, benzophenone, benzyl salicylate, ethylene brassylate, galaxolide (1,3,4,6,7,8-hexahydro-4,6,6,7,8,8-hexamethyl-cyclopenta-gama-2-benzopyran), hexyl cinnamic aldehyde, lyral (4-(4-hydroxy-4-methyl pentyl)-3-cyclohexene-10-carboxaldehyde), methyl cedrylone, methyl dihydro jasmonate, methyl-beta-naphthyl ketone, musk indanone, musk ketone, musk tibetene, phenylethyl phenyl acetate, or any combination thereof.

10. The film or sheet of claim 1, wherein the thermoplastic polymer comprises polypropylene and the odor-reducing agent comprises cyclodextrin.

11. The film or sheet of claim 1, wherein a second odor-reducing agent is applied to the surface of the film or sheet, wherein the second odor-reducing agent is the same or different than the first odor-reducing agent.

12. An insert for reducing atmospheric odor comprising the film or fibrous sheet of claim 1.

13. An article for reducing atmospheric odor comprising a film or fibrous sheet of claim 1 attached to the article.

14. The article of claim 13, wherein the article comprises vacuum bags and filters, laundry hampers, shelf or drawer liners, pet beds, lint rollers, clothing brushes, sheets, comforters, towels, electrostatic lint removers, lint traps, HVAC furnace filters, or dry cleaning bags.

15. An shaped article for removing atmospheric odors produced by the process comprising (1) extruding a first thermoplastic polymer comprising a first odor-reducing agent to produce a shaped article, and (2) applying a fragrance to the shaped article.

16. The article of claim 15, wherein the article is produced coextruding a first thermoplastic polymer and a second thermoplastic polymer, wherein the first polymer comprises the odor-reducing agent homogeneously dispersed throughout the first polymer, wherein the first and second polymer are the same or different.

17. The article of claim 16, wherein the first and second polymer comprises a poly(acrylonitrile-co-butadiene-co-styrene) polymer, an acrylic polymer, cellophane, a cellulosic, a fluoropolymer, a polyamide, a polycarbonate, a polyester, a polyimide, a polyethylene, a polypropylene, a polystyrene, a vinyl film, or any combination thereof.

18. The article of claim 16, wherein the first and second polymer comprises a polyalkylene homopolymer or copolymer.

19. The article of claim 18, wherein the polymer comprises polypropylene.

20. The article of claim 16, wherein the first odor-reducing agent comprises an inorganic compound, a hydrazine, or an organic polymer.

21. The article of claim 16, wherein the first odor-reducing agent comprises α-cyclodextrin, β-cyclodextrin, γ-cyclodextrin, or any combination thereof.

22. The article of claim 16, wherein the fragrance comprises anethole, benzaldehyde, benzyl acetate, benzyl alcohol, benzyl formate, iso-bornyl acetate, camphene, cis-citral (neral), citronellal, citronellol, citronellyl acetate, paracymene, decanal, dihydrolinalool, dihydromyrcenol, dimethyl phenyl carbinol, eucalyptol, geranial, geraniol, geranyl acetate, geranyl nitrile, cis-3-hexenyl acetate, hydroxycitronellal, d-limonene, linalool, linalool oxide, linalyl acetate, linalyl propionate, methyl anthranilate, alpha-methyl ionone, methyl nonyl acetaldehyde, methyl phenyl carbinyl acetate, laevo-menthyl acetate, menthone, iso-menthone, myrcene, myrcenyl acetate, myrcenol, nerol, neryl acetate, nonyl acetate, phenyl ethyl alcohol, alpha-pinene, beta-pinene, gamma-terpinene, alpha-terpineol, beta-terpineol, terpinyl acetate, vertenex (para-tertiary-butyl cyclohexyl acetate), amyl cinnamic aldehyde, iso-amyl salicylate, beta-caryophyllene, cedrene, cinnamic alcohol, coumarin, dimethyl benzyl carbinyl acetate, ethyl vanillin, eugenol, iso-eugenol, flor acetate, heliotropine, 3-cis-hexenyl salicylate, hexyl salicylate, lilial (para-tertiarybutyl-alpha-methyl hydrocinnamic aldehyde), gamma-methyl ionone, nerolidol, patchouli alcohol, phenyl hexanol, beta-selinene, trichloromethyl phenyl carbinyl acetate, triethyl citrate, vanillin, veratraldehyde, benzophenone, benzyl salicylate, ethylene brassylate, galaxolide (1,3,4,6,7,8-hexahydro-4,6,6,7,8,8-hexamethyl-cyclopenta-gama-2-benzopyran), hexyl cinnamic aldehyde, lyral (4-(4-hydroxy-4-methyl pentyl)-3-cyclohexene-10-carboxaldehyde), methyl cedrylone, methyl dihydro jasmonate, methyl-beta-naphthyl ketone, musk indanone, musk ketone, musk tibetene, phenylethyl phenyl acetate, or any combination thereof.

23. The article of claim 16, wherein the first and second polymer comprises polypropylene and the first odor-reducing agent comprises cyclodextrin.

24. The article of claim 16, wherein a second odor-reducing agent is applied to the surface of the article, wherein the second odor-reducing agent is the same or different than the first odor-reducing agent.

25. The article of claim 16, wherein the article comprises a storage device comprising a shelf, rack, shoe tree, hanger, hook, tray or box.

26. An article comprising a fragrance applied to the article.
